(19) United States
(12) Patent Application Publication
(10) Pub. No.: US 2002/0137029 A1
(43) Pub. Date: Sep. 26, 2002

(54) IDENTIFICATION OF NUMERICAL CHANGES IN CELL DNA

(76) Inventors: STEFAN JOOS, DOSENHEIM (DE); PETER LICHTER, GAIBERG (DE)

Correspondence Address: ALBERT P HALLUIN, HOWREY & SIMON, 1299 PENNSYLVANIA AVENUE NW, BOX NO 34, WASHINGTON, DC, 200042402

(21) Appl. No.: 09/171,854

(22) PCT Filed: Apr. 23, 1997
(86) PCT No.: PCT/DE97/00814

(30) Foreign Application Priority Data
Apr. 24, 1996 (DE) 196 16 381.1

(51) Int. Cl. C12Q 1/68

(52) U.S. Cl. 435/6

(57) ABSTRACT

The present invention relates to a process for identifying numerical changes in cell DNA, comprising the following steps:

(a) isolating DNA from normal cells and amplifying the DNA by means of a PCR method using tag primers;

(b) hybridizing cells under study in situ with the amplified DNA from (a);

(c) amplifying DNA from the in situ hybridized cells from (b) by means of a PCR method using the tag primers from (a), and

(d) identifying numerical changes in the amplified DNA from (c) in a normal way.

In addition, the invention concerns a kit suitable for carrying out the process.

### FIELD OF THE INVENTION

The present invention relates to a process for identifying numerical changes in cell DNA and a kit suitable for carrying out the process.

### BACKGROUND OF THE INVENTION

For the field of molecular tumor genetics, it is important to know which genetic changes and cytogenetic changes, are present in certain tumors, in which sequences they form and whether they are correlated with the clinical course. In order to find this out, it is necessary to investigate the DNA of small cell populations of the tumors and single-cells thereof. Many attempts have been made to achieve this goal. However, the previous results are not satisfactory.

### SUMMARY OF THE INVENTION

Therefore, it is the object of the present invention to provide a process by which it is possible to identify genetic changes and cytogenetic changes in the DNA of small cell populations and single cells. According to the invention, this is achieved by the subject matters defined in the claims.

### BRIEF DESCRIPTION OF THE DRAWINGS

FIG. 1 provides a diagram of the process according to the invention.

FIG. 2 provides chromosomal over-representations in the cell line Colo 320HSR with and without use of the “Tagged Genome Hybridization” (TGH) process according to the invention.

### DETAILED DESCRIPTION OF THE INVENTION

The present invention relates to a process for detecting numerical changes in cell DNA, which comprises the following steps:

(a) isolating DNA from normal cells and amplifying the DNA by PCR using tag primers;

(b) hybridizing of the cells under study in situ with amplified DNA from (a);

(c) amplifying DNA from the in situ hybridized cells from (b) by PCR using the tag primers from (a); and

(d) identifying numerical changes in the amplified DNA from (c) in a normal way.

The expression “normal cells” comprises cells of any kind and origin, which have no known numerical changes in their DNA.

DNA is isolated and amplified from such cells. Common methods can be used for this purpose. For the amplification, a PCR method which uses tag primers is appropriate. The expression “tag” refers to the fact that the primers can be degenerative (universal) primers, i.e., primers which can bind to many different sites of a cell DNA. Examples of such primers are DOP or SiA primers.

The expression “cells under study” comprises cells of any kind and origin. In particular, these are tumor cells or cells from the blood of pregnant persons. Cells of a small cell population or single cells are particularly preferred. Most preferred are cells having a nucleus in interphase.

The cells under study are subjected to an in situ hybridization with the amplified DNA from healthy cells. For this purpose, common conditions and materials known to those skilled in the art can be used.

The DNA from the in situ hybridized cells is used as a template for amplification. For this purpose, a PCR method which uses the above-noted tag primers is preferred.

The amplified DNA is used to determine numerical changes. For this purpose, common methods known to those skilled in the art can be used. It is preferable to carry out a comparative genomic hybridization method (CGH) (Kallioniemi A., et al., Science 258:818 (1992); Kallioniemi O. P., et al., Genes Chromosome Cancer 10:231 (1994); and Lichter P., et al., in Human Chromosomes, eds. Verma R. S. and Babu A., New York, p. 191 (1995)).

According to the invention, a kit is also provided for carrying out the process for identifying numerical changes in the DNA of cells, particularly of a small cell population or single cells. Such a kit comprises the following components:

(a) amplified DNA from healthy cells, the DNA being flanked by tag primers;

(b) tag primers, and common; and

(c) auxiliary agents, particularly those suitable for identifying numerical changes in DNA.

By means of the present invention, it is possible to identify numerical changes in the DNA of cells, particularly of small cell populations and single cells. Thus, the present invention is adapted for use in diagnosing of diseases in which the investigation of small tissue samples and cell aggregates matter. Such diseases include tumors. Furthermore, the present invention is suitable for examining embryonic cells in the blood of pregnant persons. Hence, it represents a new method for prenatal diagnostics.

The following example explains the invention.

### EXAMPLE

### Analysis of Small Cell Populations of Cell Line Colo 320(HSR) by the Process According to the Invention

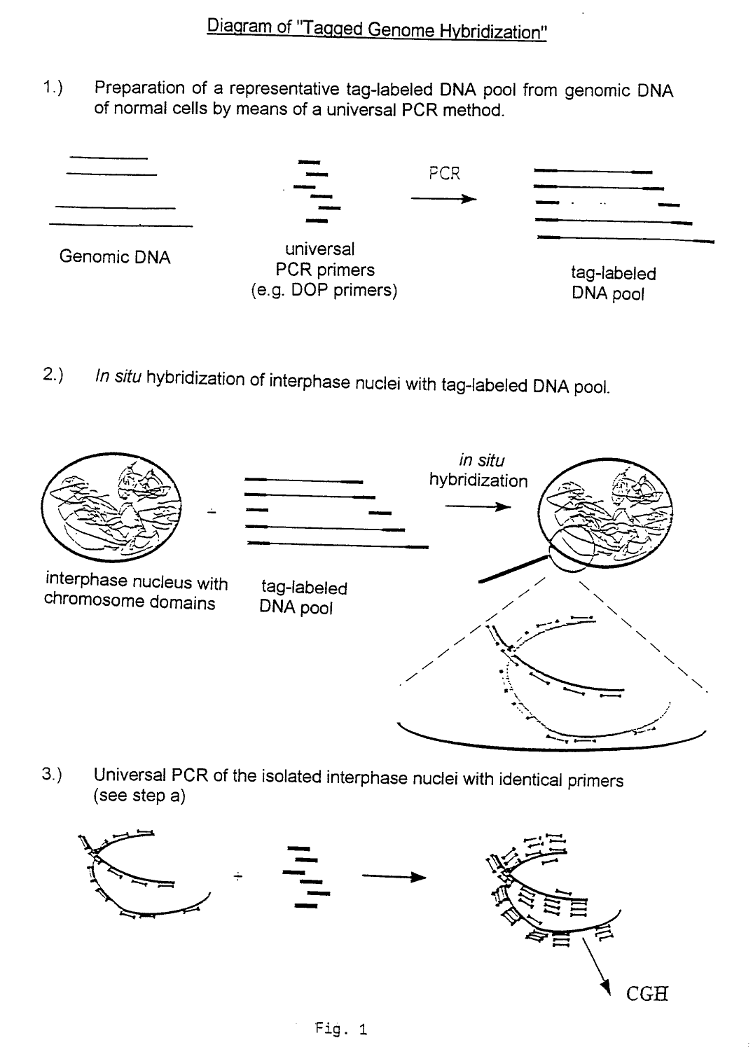

The process, according to the invention (the TGH process of FIG. 1), is carried out below with cell line Colo 320 (HSR). The TGH process proceeds in several individual steps as follows:

Prepare Colo 320(HSR) interphase nuclei on slides.

Prepare a tag-labeled sequence pool of normal genomic DNA by means of DOP PCR.

Modify the tag-labeled genomic DNA.

Hybridize the tag-labeled genomic DNA on interphase nuclei of cell line Colo 320(HSR) in situ.

Isolate Colo 320(HSR) cell populations of defined number by micromanipulation. To elucidate the efficiency of the TGH treatment, both cell nuclei hybridized with tag-labeled DNA and non-hybridized cell nuclei were isolated and then compared.

Amplify the DNA of the isolated cell nuclei by means of DOP PCR.

Perform comparative genomic hybridization (CGH) to identify chromosomal imbalances of Colo 320(HSR) cells.

As far as preparing the interphase nuclei, isolating genomic DNA from blood and the CGH protocol are concerned, reference is made to known methods (Kallioniemi A., et al., Science 258:818 (1992); Kallioniemi O. P., et al., Genes Chromosome Cancer 10:231 (1994); and Lichter P., et al. in Human Chromosomes, eds. Verma R. S. and Babu A., New York, p 191 (1995)). The teachings of these references are herein incorporated by reference.

Step I

Preparation of a tag-labeled sequence pool of normal genomic DNA by means of DOP PCR.

Reagents, Buffers and Solutions:

10× reaction buffer: 20 mM MgCl₂; 500 mM KCl; 100 mM Tris-HCl (pH 8.4); 1 mg/ml gelatin

10× nucleotide-mix: 2 mM of each nucleotide dATP, dCTP, dGTP, dTTP (prepared from the deoxynucleoside triphosphate set from Boehringer Mannheim, Germany, Catalog No. 1277049).

L0× DOP primer: 20 μM of oligonucleotide 5′ CCG ACT CGA GNN NNN NAT GTG G3′, (N=A, C, G or T).

For minigels: agarose; TBE buffer: 0.089 M Tris-borate, 0.089 M boric acid, 2 mM EDTA (pH 8.0); suitable size marker (e.g., 1 kb marker; catalog No. 15615-016; Gibco BRL, Eggenstein, Germany); gel loading buffer having 0.25% bromophenol blue and 30% glycerol.

Starting DNA of normal cells, to be labeled and amplified.

Detailed Course:

1. The following components were pipetted on ice into a 500 μl reaction vessel (PCR vessels having integrated volume restriction; Sarstedt, Nümbrecht, Germany) (total volume 50 μl):

100 ng genomic starting DNA;

5 μl 10× reaction buffer;

5 μl 10× nucleotide mix;

5 μl 10× DOP primer;

H₂O add to 50 μl; and

1.25 U taq polymerase (should be added last).

2. The same components were transferred with a pipette as negative control but without the addition of the starting DNA.

3. A device from Omnigene/Hybaid (Type AZ 1623; supplier MWG Biotech Ebersbach near München, Germany) was used for the PCR reactions. The following temperature program was employed:

initial denaturation of the starting DNA by heating to 93° C. for 10 min.

5 cycles (what is called “low stringency phase”) each with: 94° C., 1 min.; 30° C., 3 min.; 30° C.-72° C., 3 min.; 72° C., 3 min.

35 cycles (what is called “high stringency phase”) each with: 94° C., 1 min.; 62° C., 1 min.; 72° C., 3 min. (with an extension of 1 sec/cycle).

a final extension step at 72° C. for 10 min.

4. After the PCR reaction, an aliquot of 7 μl of the product was separated on an agarose gel. A 1 kb marker served as the size marker. The separation was made in 1× TBE buffer with 100 V for 30 min. The applied DNA was made visible by staining using ethidium bromide and photographed. A “smear” with DNA fragments between 200 base pairs and 2000 base pairs was usually visible. No DNA was identifiable within this size range in the negative control.

5. Finally, the rest of the nucleotides were separated from the tag-labeled DNA by means of a PCR purification kit (Diagen GmbH, Hilden, Germany, catalog No. 28106).

Step 2

Modification of the tag-labeled genomic DNA for the in situ hybridization.

Reagents, Buffers and Solutions:

10× reaction buffer with 0.5 M Tris-HCl (pH 8.0), 50 mM MgCl₂, 0.5 mg/ml bovine serum albumin (fraction V, catalog No. 735078, Boehringer Mannheim).

0.1 M β-mercaptoethanol.

10× nucleotide stock solution with 0.5 mM dATP, 0.5 mM dCTP, 0.5 mM dGTP, 0.125 mM digoxigenin-11-dUTP (Boehringer Mannheim, catalog No. 1558706), 0.375 mM dTTP.

Escherichia coli-DNA-polymerase I (New England Biolabs GmbH, Schwalbach/Taunus, Germany, catalog No. 209L).

DNase I stock solution: 3 mg in 1 ml 0.15 M NaCl, 50% glycerol.

Column buffer: 10 mM Tris-HCl (pH 8.0), 1 mM EDTA, 0.1% SDS.

Columns: Sephadex GSO (medium), 1 ml tuberculin syringes (e.g., Primoll from Pharmaplast A/S, DK-4970 Rodby), glass wool.

For Minigel: Agarose; TBE buffer; 1 kb size marker (Gibco BRL, Eggenstein, catalog No. 15615-016); gel loading buffer (see above).

Detailed Course:

1. 2 μg of the tag-labeled DNA (see step 1) were transferred with a pipette into a reaction vessel together with 10 μg of the 10× reaction buffer, 10 μl β-mercaptoethanol, 10 μl of the nucleotide stock solution, 20 U of DNA-polymerase I, and 2 μl of a 1:1000 dilution of the DNase stock solution (in water).

2. The modification reaction was carried out at 15° C. for 30-40 min.

3. The reaction mixture was placed on ice and an aliquot was tested for the fragment size suitable for the in situ hybridization.

4. The fragment length of the DNA was determined by gel electrophoresis. 10 μl of the reaction batch were admixed with 3 μl gel loading buffer and denatured in the boiling water bath for 2-3 min. After another 3 min. on ice, the aliquot was applied to a 1-2% agarose minigel, together with the 1 kb size marker. The DNA fragments were separated with 15 V/cm for 30 min. After staining using ethidium bromide, the gel could be photographed under U.V. light and the size of the DNA fragments could be determined.

5. In the optimum case, the DNA fragments should have a size range between 500 and 1000 base pairs. When the average size of the fragments was above this range, the remaining DNA was incubated once again with DNase I until the optimum size of the fragments was reached.

6. In order to inactivate the DNase, 2 μl of 0.5 M EDTA (final concentration 10 mM) and 1 μg 10% SDS (final concentration 0.1%) were added to the batch and the reaction batch was heated at 68° C. for 10 min.

7. Non-incorporated nucleotides were separated from the DNA sample by gel filtration using separation columns, which had been produced as follows.

(a) A 1 ml tuberculin syringe was initially packed with glass wool up to the 0.2 ml mark and then filled with buffered Sephadex G50 up to the 1 ml mark. The column was transferred into a 15 ml vessel and centrifuged at 2000 g and room temperature for 6 min.

(b) After adding 100 μl of column buffer each, the columns were centrifuged again. This step was repeated three times.

(c) The DNA was placed on the column, centrifuged (2000 g, 6 min.) and collected in a reaction vessel. The final concentration of this DNA sample is about 20 ng/μl. It can be stored at −20° C. over a prolonged period of time (months to years).

Step 3

In situ hybridization of the tag-labeled genomic DNA on interphase nuclei of line Colo 320(HSR).

A. Denaturation of the Genomic DNA of the Colo 320 (HSR) Interphase Nuclei

Reagents, Buffers and Solutions:

Denaturation solution: 70% deionized formamide (for molecular biology, catalog No. 112027, Merck, Darmstadt, 2× SSC, 50 mM sodium phosphate (pH 7 with 1 M HCl).

ethanol (ice-cold): 70%, 90% and 100%.

Detailed Course:

1. The denaturation solution was placed in a cuvette and heated in a water bath to 70° C.

2. The cover glasses (76×26 mm) including the Colo 320(HSR) cell nuclei under study were incubated in this solution for 2 min. precisely and then immediately transferred into the cold 70% alcohol.

3. The cover glasses were dehydrated in 70%, 90% and 100% ethanol for 5 min. each and then air-dried.

B. Precipitation and Denaturation of the Tag-labeled DNA Sample for the in situ Hybridization.

Reagents, Buffers and Solutions

3 M sodium acetate, pH 5.2.

Deionized formamide (for molecular biology, catalog No. 112027, Merck, Darmstadt). The deionization was made by stirring the formamide with an ion exchanger (e.g., AG 501-X8 (D) Resin, catalog No. 142-6425 of Biorad, München, Germany).

2× hybridization buffer: 4× SSC, 20% dextransulfate.

Detailed Course:

1. 1 μg of the tag-labeled DNA and 50 μg of human Cotl DNA (Gibco-BRL, Eggenstein, catalog No. 5279SA) were precipitated by adding 1/20 volume of 3 M sodium acetate and 2.5 volumes of 100% ethanol.

2. After centrifuging at 12,000 rpm and 4° C. for 10 min., the supernatant was separated, the pellet was washed with 500 μl 70% ethanol, centrifuged again (12,000 rpm, 10 min., 4° C.) and then lyophilized.

3. The precipitated DNA was inserted in 6 μl deionized formamide and shaken at room temperature for 30 min. (Vortex).

4. After adding 6 μl of 2× hybridization buffer, shaking was carried out again for 30 min.

5. The DNA was denatured at 75° C. for 5 min., placed on ice for 5 min. and then incubated at 37° C. for 30 min. (“preannealing”).

C. En situ Hybridization.

Detailed Course:

1. 12μ of the hybridization mix including the denatured DNA were placed on a large cover glass comprising the cell nuclei under study.

2. A cover glass having a size of 18×18 mm was placed thereon.

3. The 18×18 mm cover glass was sealed with liquid adhesive, and the preparation was incubated in a moist chamber at 37° C. for 48 h.

D. Washing and Detection of the Hybridized Cell Nuclei.

Reagents, Buffers and Solutions:

Wash solution A: 50% formamide (p.A. catalog No. 9684, Merck, Darmstadt), 2× SSC.

Wash solution B: 0.1× SSC.

Wash solution C: 4× SSC, 0.1% Tween 20.

Wash solution D: 2× SSC, 0.05% Tween 20.

Blocking solution: 3% BSA, 4× SSC, 0.1% Tween 20.

Detection buffer: 1% BSA, 4× SSC, 0.1% Tween 20.

Anti-digoxigenin rhodamine, FAB fragments (catalog No. 1207750, Boehringer Mannheim)

Antifade solution: 0.233 g DABCO (1,4-diazabicyclo-2,2,2-octane), 20 mM Tris-HCl, pH 8.0, 90% glycerol.

4,6-diamino-2-phenylindole (DAPI)

Detailed Course:

1. Wash solutions A and B were heated in a water bath in a cuvette to 42° C.

2. The seal ring of liquid adhesive was removed from the cover glass which was then washed in wash solution A at 42° C. for 3×10 min.

3. Thereafter, washing was carried out in wash solution B for 3×10 min.

4. 200 μl of the blocking solution were pipetted onto the cover glass, a cover glass was again placed thereon and incubated at 37° C. in a moist chamber for 30 min.

5. The blocking solution was removed; 200 μl of the detection buffer having 6 μg/ml rhodamine-conjugated anti-digoxigenin (Boehringer, Mannheim) were placed on the cover glass instead and incubated under a cover glass at 37° C. in a moist chamber for 30 min.

6. The large cover glass with the cell nuclei was then washed in wash solution C at 42° C. three times for 10 min. each.

7. Thereafter, the large cover glass with the cell nuclei was incubated in 2× SSC in which 200 ng/ml (DAPI) were dissolved for 20 min.

8. Washing of the cover glass in wash solution D at room temperature for 1 to 2 min.

9. Covering the hybridized interphase nuclei with antifade solution.

Step 4

Isolation of the Hybridized Nuclei by Micromanipulation.

Apparatus:

Microscope Axioskop FS (Carl Zeiss, Jena, Germany) equipped for fluorescence microscopy.

De Fonbrune micromanipulator (Bachofer, Reutlingen, Germany).

De Fonbrune “microforge” (Mikroschmiede) (Bachofer, Reutlingen) for the production of microtips.

Glass plates from B270: size 70×35 mm, thickness 6 mm; with step: width 26 mm, depth 4 mm. (Produced by Berliner Glas KG, Waldkraichburger Straβe 5, 12347 Berlin, Germany, order No. B 100158-PO/e h).

Detailed Course:

1. The large cover glass with the Colo cell nuclei to be isolated was placed on the B270 glass plate (with the cells facing downwards); the intermediate space between base bottom and cover glass (and cell nuclei, respectively) was filled with antifade solution.

2. A microtip produced in the microforge was inserted in the micromanipulator. The microtip was extended by means of the microforge such that it faced upwards at an angle of about 30%.

3. The microtip was then pushed into the intermediate space filled with the antifade solution, so that its lower portion reached the cell nuclei to be isolated.

4. The Colo nuclei could be made visible under fluorescent conditions by a suitable filter (filter set No. 487915-9901 for rhodamine or filter set No. 487901-9901 for DAPI, Carl Zeiss, Jena) under the microscope. By using transmitted light, it was also possible to simultaneously recognize the microtip. In this way, it was possible to “pick up” the cell nuclei individually onto the microtips. (Remark: Since the cell nuclei are not brittle in the antifade solution but resilient, they can be isolated as a whole).

5. Having picked up one nucleus each with a needle, the latter was broken off at the bottom of a PCR tube filled with 20 μl H₂O. In this way, populations having an accurately defined cell number of hybridized and non-hybridized nuclei could be isolated. (Remark: The non-hybridized cell nuclei had been treated in the same way as the hybridized nuclei, but in the in situ hybridization no tag-labeled DNA had been added).

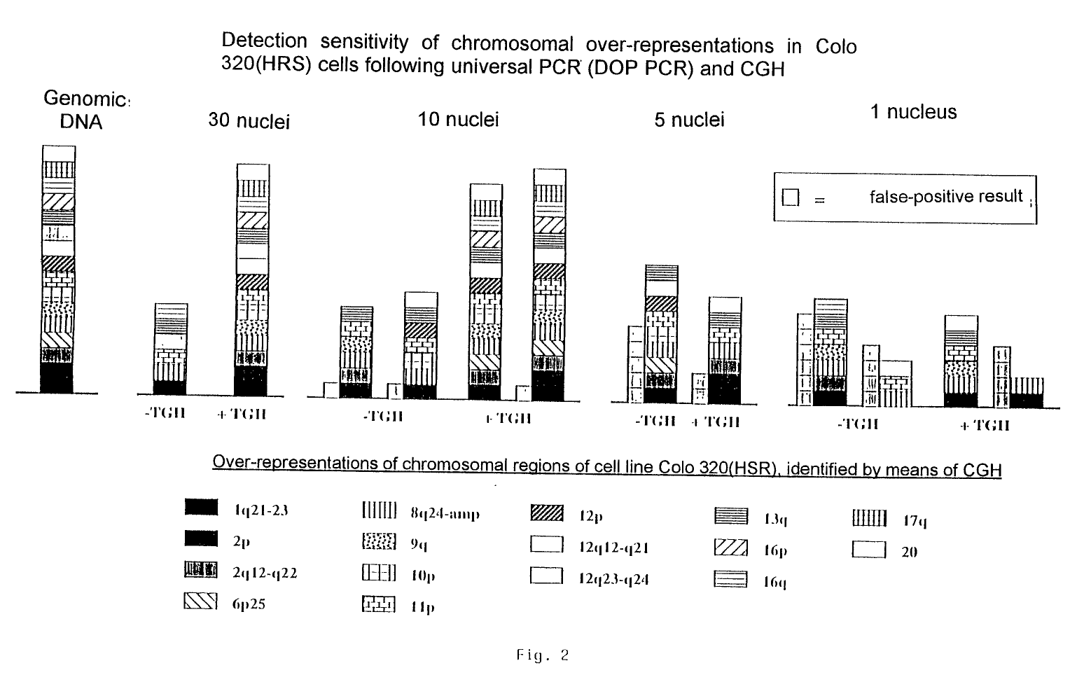

Following this step, the isolated nuclei were subjected to DOP PCR. This protocol is identical with step 1 (points 15), however, no further starting DNA is added with the exception of the isolated nuclei. The DOP PCR product was used for common CGH analysis (Kallioniemi A., et al., Science 258:818 (1992); Kallioniemi O. P., et al., Cenes Chromosome Cancer 10:231 (1994); and Lichter P., et al. in Human Chromosomes, eds. Verma R. S. and Babu A., New York, p. 191 (1995)). It showed that by means of the process according to the invention between 89% and 94% of all chromosome over-representations were recognized in 30 Colo 320(HSR) cells and 10 Colo320 (HSR) cells, respectively, whereas it was only between 38% and 44% without the TGH process. In addition, the number of false-positive findings in the TGH-treated group is markedly lower than that of the non-TGH-treated group (7 over 18) (FIG. 2).

1. A process for detecting numerical changes in cell DNA, comprising the following steps:
(a) isolating DNA from cells which have no known numerical changes in their DNA, and amplifying the DNA by means of a PCR method using tag primers;
(b) hybridizing of cells under study in situ with the amplified DNA from (a);
(c) amplifying DNA from the in situ hybridized cells from (b) by means of a PCR method using the tag primers from (a); and
(d) identifying numerical changes in the amplified DNA from (c).

2. The process according to claim 1, wherein the cells under study originate from tumors.

3. The process according to claim 1, wherein the cells under study originate from the blood of pregnant persons.

4. The process according to claim 1, wherein the cells under study are those of a small cell population or single cells.

5. The process according to claim 1, wherein the cells under study have a nucleus in interphase.

6. The process according to claim 1, wherein the tag primers are degenerative primers.

7. The process according to claim 1, wherein the identifying is performed by a “Comparative Genomic Hybridization” (CGH) method.

8. A kit for carrying out the process according to claim 1, comprising the following components:
(a) amplified DNA from cells that have no known numerical changes in their DNA, the DNA being flanked by tag primers;
(b) tag primers; and
(c) auxiliary agents for identifying numerical changes in a DNA.
